(19) United States
(12) Patent Application Publication
(10) Pub. No.: US 2021/0345967 A1
(43) Pub. Date: Nov. 11, 2021

(54) PHYSIOLOGICAL MONITORING SYSTEM FOR A NEONATE AND A NEONATAL BLANKET POWERING A WIRELESS PHYSIOLOGICAL SENSOR

(71) Applicant: GE Precision Healthcare LLC, Wauwatosa, WI (US)

(72) Inventor: Steven J. Woloschek, Wauwatosa, WI (US)

(73) Assignee: GE Precision Healthcare LLC, Wauwatosa, WI (US)

(21) Appl. No.: 16/870,106

(22) Filed: May 8, 2020

(51) Int. Cl. A61B 5/00; A61B 5/0408; H04B 5/00; H01Q 1/38

(52) U.S. Cl. CPC A61B 5/6887; A61B 5/0006; A61B 5/04085; A61B 2560/0214; H01Q 1/38; A61B 2503/045; H04B 5/0037

(57) ABSTRACT

A neonatal blanket includes a substrate material configured to cover a neonate and at least one antenna on the substrate material, where the antenna is configured to transmit RF energy to power the at least one wireless physiological sensor on the neonate. The blanket further includes a power connection configured to connect to a power source to power the wireless physiological sensor via the antennae.

### BACKGROUND

The present disclosure generally relates to physiological monitoring systems for neonates, and more particularly to systems and methods for powering wireless physiological sensors on a neonate.

Neonates, particularly premature infants, are often placed within an incubator or a warmer system so that they may have a controlled and monitored environment to aid in their survival and growth. Typically, such neonates require monitoring by physiological sensors. These physiological sensors are typically wired to an incubator or warmer, or to a monitoring device placed on or near the incubator/warmer system. Neonatal incubators, warmers, and other neonatal care systems may include integrated physiological monitoring systems, or monitoring may be done by one or more separate patient monitoring devices. Depending on the physiological parameter being monitored, such monitoring may be conducted continuously or periodically. However, physiological monitoring of the infant is often interrupted, such as when the neonate is moved out of the infant care device for care or treatment, and thus the physiological sensors must be unplugged from the monitoring system.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a neonatal blanket includes a substrate material configured to cover a neonate and at least one antenna on the substrate material, where the antenna is configured to transmit RF energy to power the at least one wireless physiological sensor on the neonate. The blanket further includes a power connector configured to connect to a power source to power the wireless physiological sensor via the antenna.

One embodiment of a physiological monitoring system for a neonate includes at least one wireless physiological sensor configured to record physiological information from a neonate, and a neonatal blanket. The neonatal blanket comprises a substrate material and at least one antenna on the substrate material, wherein the antenna is configured for wireless communication with and transmission of RF energy to power at least one wireless physiological sensor when the neonate is covered by the blanket. A control circuit is configured to drive RF power to the antenna and configured to receive physiological data from the wireless physiological sensor via the antenna. A wireless transmitter is configured to wirelessly transmit the physiological data to a device configured to receive patient monitoring data for the neonate. A battery is configured to power the at least one wireless physiological sensor via the antenna and is also configured to power the wireless transmitter.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 depicts one embodiment of a physiological monitoring system for neonate in accordance with the present disclosure.

FIG. 2 is a schematic illustration of a physiological monitoring system for a neonate according to one embodiment of the present disclosure.

FIG. 3 a neonatal blanket according to one embodiment of the disclosure.

### DETAILED DESCRIPTION

The inventors have recognized that wireless physiological sensors have significant advantages in neonatal monitoring. The problems of dealing with wires while caring for the neonate can be avoided. Wired sensors may be obstructive while caring for an infant, for example, and thus monitoring may need to be interrupted and sensors unplugged from the care device or patient monitoring during certain infant care tasks or procedures. Further, wireless physiological sensors offer an opportunity for continuous monitoring of a neonate while a neonate is being treated by a clinician and/or moved out of the infant care device, such as an incubator or warmer. Neonates are often moved out of and away from the care device for checkups, procedures, other treatments, family visits, etc. Thus, continuous monitoring of the infant is often impossible with wired sensors, as the wired connections are severed when the neonate is not in the care device. Wireless sensors, on the other hand, provide the ability to conduct monitoring as the patient is moved in and out of the infant care device and during procedures where wires might otherwise be in the way.

The inventors have recognized that physiological sensors on neonates, including premature neonates, must be very small and lightweight. Given the small size of neonates, particularly premature neonates who are in most need of continuous monitoring, the sensors must not be too large or heavy in order to be viable for long term use on the neonate. Batteries are often the heaviest elements on wireless sensing devices, and thus the inventors have endeavored to devise a system to power physiological sensors on a neonate that is easy to use within the workflow of neonatal care. The disclosed neonatal physiological monitoring system is configured to wirelessly power one or more wireless physiological sensors on a neonate via one or more antennae incorporated on a neonatal blanket. Thereby, batteries can be eliminated from the wireless physiological sensors, making them much lighter and smaller without compromising functionality or usability.

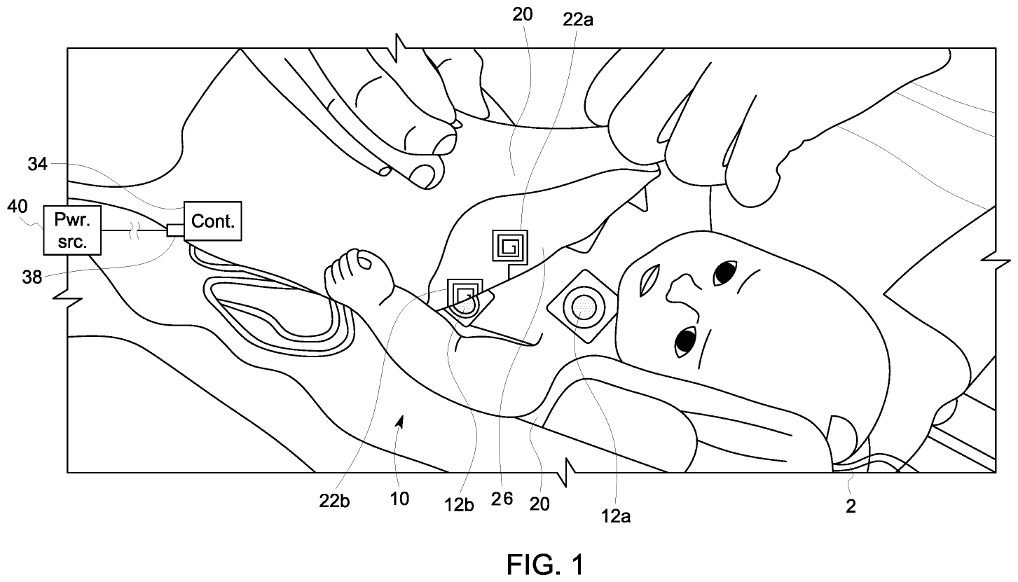

FIG. 1 depicts an exemplary embodiment of a neonatal physiological monitoring system 10 comprising a neonatal blanket 20 configured to power wireless physiological sensors 12 on the neonate 2. In some embodiments, the neonatal blanket 20 includes at least one antenna 22 configured to both power and wirelessly communicate with one or more physiological sensors 12 on the neonate 2, such as to receive physiological data therefrom. The blanket 20 comprises a substrate material that covers the neonate 2. Multiple antennae 22 are on the substrate material of the blanket 20. Each antenna 22 (e.g., 22a and 22b) is configured to transmit RF energy to power at least one wireless physiological sensors 12 (e.g., 12a and 12b) on the neonate 2. In one such embodiment, each antenna 22a and 22b is configured to power and/or receive data from one physiological sensor 12a or 12b in a one-to-one relationship between antennae and sensors.

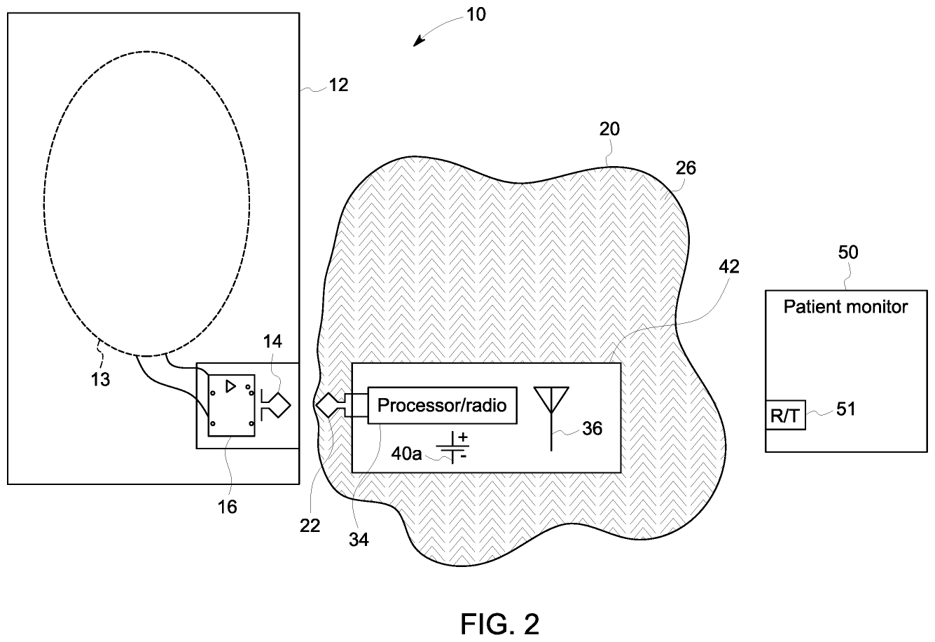

Each antenna 22 is connected to and receives power from a power source 40, such as via power connection 38 on or connected to the blanket 20. The power source 40 may be any of the various types of power sources. In one embodiment, the power source 40 is a battery 40a (see FIG. 2) on or associated with the blanket 20. Thereby, the neonatal blanket system 20 and the physiological monitoring system 10 are portable. Alternatively or additionally, the blanket 20 may be configured to connect to an external power source 40, such as via a power cord and the power connection 38. For example, the power connection 38 may be a universal serial bus (USB) receiver, such as a USB-C connector, connectable via a USB cable to a power source 40 in a neonatal care device, such as an incubator or warmer, or on a patient monitor 50. Alternatively, the power connection 38 may be an AC connection, such as to wall power outlet. Other power sources and power connection arrangements are in the art and within the scope of the present disclosure.

With reference also to FIG. 2, the neonatal blanket 20 may further include a control circuit 34 configured to drive RF power to the antenna. The control circuit 34 may also be configured to communicate with the physiological sensor, such as to receive physiological data from the wireless physiological sensor 12 via the antenna 22. For example, the control circuit 34 and the antenna 22 may comprise a near field communication (NFC) circuit where the antenna 22 is an NFC antenna 22 and the control circuit 34 includes an NFC integrated circuit and other standard NFC circuit components. The NFC circuit is configured to transmit RF energy to and communicate with the physiological sensor 12 when the antenna 22 is within a short distance, such as several centimeters, of the physiological sensor 12.

Similarly, the antennae 22 and 14 configured to communicate via NFC standard communication may be configured to transmit the physiological data recorded by the sensor 12 from the antenna 14 to the antenna 22 on the blanket 20. In such embodiments, the control circuit 34 may be configured to receive and transmit the physiological data to a patient monitoring device 50 or other device configured to receive patient monitoring data for the neonate. The neonatal blanket may further comprise a wireless transmitter 36 (e.g., a wireless transceiver) configured to transmit the physiological data to a third device or system, such as patient monitor 50. For example, the wireless transmitter 36 may be configured to transmit the physiological data via a second protocol other than NFC, such as Bluetooth, Bluetooth Low Energy (BLE), Zigbee, ANT, Wi-Fi, etc. The second protocol executed by the wireless transmitter 36 is appropriate for longer-range transmission, such as several feet or even several hundred feet.

In certain embodiments, the control circuit 34 is configured to process the physiological data received from the wireless sensor 12 to create processed physiological data. The processing may include, for example, value determinations based on the physiological data, such as calculation of heart rate, respiration rate, SpO2, temperature, blood pressure, or any other physiological parameter based on the physiological data received from the sensor 12 or from a group of sensors. Each wireless physiological sensor 12 on the neonate may be in communication with a respective antenna 22 on the blanket 20. FIG. 1 provides one such example, where each ECG sensor 12a and 12b communicates with a respective antenna 22a and 22b. The control circuit 34 may be configured to receive the physiological data, such as the ECG potentials, recorded at each of the wireless sensors 12 and to process that data to generate processed physiological data—such as ECG waveforms and/or heart rate based on the ECG potentials recorded at each of the ECG sensors 12a and 12b.

The control circuit 34 may be configured to transmit the processed physiological data via the wireless transmitter 36 in addition to or in place of the raw physiological data received from the physiological sensor 12. In other embodiments, the control circuit 34 may incorporate a storage device and may be configured to store the physiological data received from the sensor 12. In one embodiment, the blanket 20 may be configured to connect to a patient monitor 50, care device (e.g., incubator or warmer), or other device configured to receive patient monitoring data for the neonate, such as via USB or other data transfer connection. The blanket 20 may be configured to continue communication and powering the physiological sensors 12 upon disconnection from the data transfer connect for a short duration, such as during transport of the neonate, and may be configured to store the physiological data recorded for the neonate during that disconnected duration. The control circuit 34 may be configured to transfer the stored physiological data upon reconnection to the patient monitor 50 or other device configured to receive patient monitoring data for the infant.

The blanket 20 may include a battery power source 40a connected to and/or integrated into a module 42 on or connected to the substrate 26 on the blanket 20. For example, the module housing 42 encapsulating the control circuit 34. The module housing 42 may further encapsulate the wireless transmitter 36 and/or the battery 40a. In some embodiments, the housing 42 may be plastic or other material appropriate for protecting the electronic devices from damage by handling or egress of fluids, as well as to help insulate from electrical interference.

The electrical sensor 12 may be any type of physiological sensor configured for recording or otherwise obtaining physiological data from the neonate 2. For example, the physiological sensor may be an ECG sensor, an SpO2 sensor, a temperature sensor, a respiration sensor, any other type of sensor available for obtaining physiological data from the neonate 2. The physiological sensor 12 may include an electrode 13 or other detector or sensing element for recording or obtaining physiological data from the neonate 2. The wireless physiological sensor 12 further includes an on-sensor control circuit 16 connected to the antenna 14 and configured to receive RF energy and/or communicate with the control circuit 34 on the blanket 20. The on-sensor control circuit 16 is configured to digitize the physiological data prior to transmission and thus, comprises an analog to digital converter. The control circuit 16 may further be configured to perform certain data preprocessing tasks, such as filtering and/or amplification prior to transmission of the physiological data to the blanket 20.

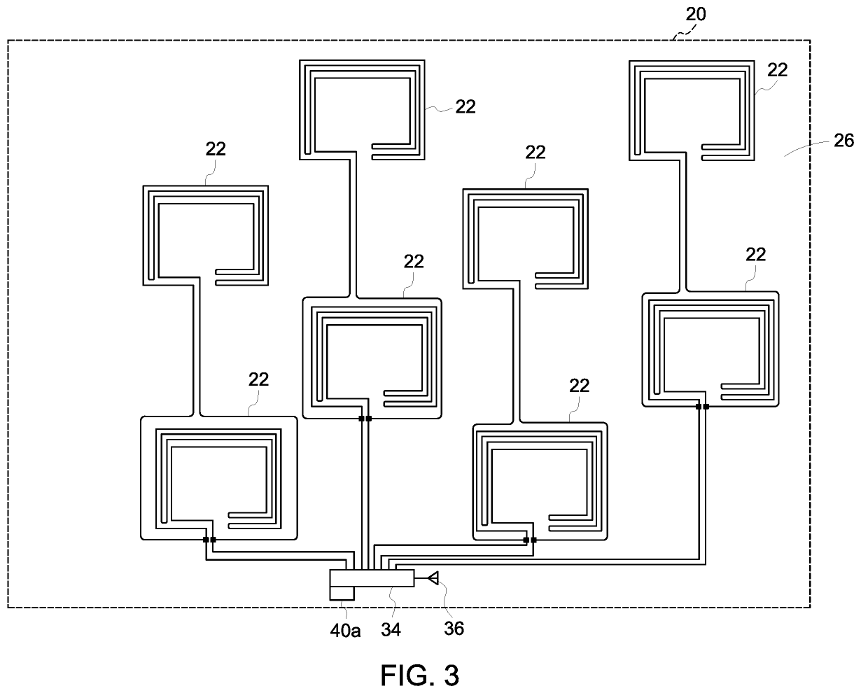

FIG. 3 is a schematic diagram of an exemplary embodiment of a blanket 20. The blanket 20 includes a substrate 26 shaped, sized, and configured to cover the neonate 2. In certain embodiments, the substrate 26 may be shaped, sized, and configured to be wrapped around the neonate, such as sufficiently large to cover the circumference of the neonate and longer than the neonate such that the neonate's feet and head are also covered. In certain embodiments, the substrate 26 may be rectangular or square. In other embodiments, the substrate 26 may be an oval or another oblong shape. In still other embodiments, the substrate 26 may be shaped and configured to swaddle the neonate, such as having side portions that wrap around the neonate and can be secured around the neonate for swaddling. In some embodiments, the blanket 20 may include fastening elements to facilitate swaddling, such as Velcro. The substrate 26 may be comprised of any of various materials suitable as a neonatal blanket such as cotton or another textile. Alternatively or additionally, the substrate 26 may be comprised of a paper material, or may be comprised of a synthetic material such as polyester. In certain embodiments, the blanket 20 may be comprised of two or more layers of material, where one or more antennae 22 are on at least one of the layers. For example, antennae 22 may be adhered to a backside of the substrate material 26, and a second layer of material may cover the backside so as to protect the antennae 22 and to protect the neonate 2 from having the antennae 22 contacting skin.

One or more antennae 22 are on the substrate material 26, such as adhered to or integrated into the substrate material. In one embodiment, the antennae 22 may be adhered to the substrate 26 by printing conductive ink onto the substrate 26. In another embodiment, the antennae 22 may be formed by silk-screening a conductive material onto the substrate 26. In still other embodiments, the antennae 22 may be formed by gluing or otherwise adhering conductive traces, such as a foil or wire, onto the substrate 26. In still other embodiments, the antennae 22 may be formed by sewing or weaving conductive fibers into the substrate 26, such as a metallic thread sewn onto or woven into the material the concentric shape illustrated in FIG. 3.

The blanket 20 may include various numbers of one or more antennae 22. In one embodiment, at least two antennae at different locations on the substrate 26, such as arranged on the substrate such that they align with locations, or potential locations, of sensors on the neonate when the blanket 20 covers or is wrapped around the neonate 2. FIG. 3 illustrates one embodiment having multiple antennae 22 arranged around the blanket such that sufficient coverage is provided across the blanket 20 so that one or more wireless sensors 12 will be powered when the blanket 20 is placed on or wrapped around the neonate. FIG. 3 provides just one exemplary arrangement of antennae, and many other arrangements may be provided and are considered within the scope of the present disclosure. For example, the antennae 22 may be placed at key locations on the blanket 20 that are likely to align with the locations of wireless physiological sensors on the neonate 2 when the blanket 20 is covering the neonate 2 or is wrapped around the neonate 2.

In certain embodiments, the blanket 20 may include multiple antennae 22 arranged on the substrate such that they are likely to align with locations of various types of wireless physiological sensors 12 on the neonate. For instance, as demonstrated in FIG. 1, certain antennae 22a and 22b may be arranged on the substrate 26 such that they align with wireless ECG sensors 12a and 12b, respectively, on the neonate when the blanket 20 is wrapped around the neonate 2. Similarly, antennae 22 may be placed on the substrate 26 at likely locations for other types of sensors, such as SpO2 sensors, temperature sensors, etc., depending on where those sensor devices are typically attached onto a neonate.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. A neonatal blanket comprising:
a substrate material configured to cover a neonate;
at least one antenna on the substrate material, wherein the antenna is configured to transmit RF energy to power at least one wireless physiological sensor on the neonate; and
a power connection configured to connect to a power source to power the wireless physiological sensor via the antenna.

2. The neonatal blanket of claim 1, further comprising at least two antennae at different locations on the substrate, wherein each antenna is configured to transmit RF energy to power one wireless physiological sensor on the neonate that is within range of the antenna.

3. The neonatal blanket of claim 2, further comprising multiple antennae at different locations on the substrate.

4. The neonatal blanket of claim 3, wherein the substrate is shaped to be wrapped around the neonate and wherein the multiple antennae are arranged on the substrate such that at least a portion of the multiple antennae align with locations of ECG sensors on the neonate when the substrate is wrapped around the neonate.

5. The neonatal blanket of claim 1, wherein the at least one antenna is further configured for wireless communication with at least one wireless physiological sensor on the neonate.

6. The neonatal blanket of claim 1, wherein the at least one antenna is an NFC antenna.

7. The neonatal blanket of claim 1, further comprising a control circuit configured to drive RF power to the antenna and configured to receive physiological data from the wireless physiological sensor via the antenna.

8. The neonatal blanket of claim 7, wherein the control circuit is further configured to process the physiological data received from the wireless physiological sensor to generate processed physiological data and to transmit the processed physiological data to a device configured to receive patient monitoring data for the neonate.

9. The neonatal blanket of claim 7, further comprising a wireless transmitter that wirelessly transmits the processed physiological data to a device configured to receive patient monitoring data for the neonate.

10. The neonatal blanket of claim 9, wherein the at least one antenna is an NFC antenna and the wireless transmitter configured to transmit via a second wireless transmission protocol other than NFC.

11. The neonatal blanket of claim 1, wherein the at least one antenna comprises conductive ink printed on the substrate material.

12. The neonatal blanket of claim 1, wherein the substrate is a textile and wherein the at least one antenna comprises conductive fibers woven into or sewn on the textile.

13. The neonatal blanket of claim 1, further comprising a battery connected to a control circuit via the power connection and configured to power the antenna.

14. A physiological monitoring system for a neonate, the system comprising:
at least one wireless physiological sensor configured to record physiological information from a neonate;
a neonatal blanket comprising a substrate material and at least one antenna on the substrate material, wherein the antenna is configured for wireless communication with and transmission of RF energy to power at least one wireless physiological sensor when the neonatal blanket is on the neonate;
a control circuit on the blanket configured to drive RF power to the antenna and configured to receive physiological data from the wireless physiological sensor via the antenna;
a wireless transmitter configured to wirelessly transmits the physiological data to a device configured to receive patient monitoring data for the neonate; and
a battery configured to power the at least one wireless physiological sensor via the at least one antenna and the control circuit and to power the wireless transmitter.

15. The system of claim 14, further comprising at least two antennae adhered at different locations on the substrate, wherein each antenna is configured to transmit RF energy to power wireless physiological sensor on the neonate that is within range of the antenna.

16. The system of claim 14, further comprising multiple antennae arranged on the substrate such that at least a portion of the multiple antennae align with locations of ECG sensors on the neonate when the substrate is wrapped around the neonate.

17. The system of claim 14, wherein the at least one antenna is an NFC antenna.

18. The system of claim 17, wherein the wireless transmitter configured to transmit via a second wireless transmission protocol other than NFC.

19. The system of claim 14, wherein the control circuit is further configured to process the physiological data received from the wireless sensor to generate processed physiological data, and to operate the wireless transmitter to transmit the processed physiological data to a device configured to receive patient monitoring data for the neonate.

20. The system of claim 14, wherein the at least one antenna comprises conductive ink printed onto the substrate material.
